(19) United States
(12) Patent Application Publication
(10) Pub. No.: US 2022/0282195 A1
(43) Pub. Date: Sep. 8, 2022

(54) MOBILE PROCESSING UNIT AND LABORATORIES

(71) Applicant: ORGENESIS INC., Germantown, MD (US)

(72) Inventors: Gilad Ish Shalom, Ramat Hakovesh (IL); Inbar Barzilay, Nahariya (IL); Vered Caplan, Tel Aviv (IL)

(73) Assignee: ORGENESIS INC,, Germantown, MD (US)

(21) Appl. No.: 17/689,845

(22) Filed: Mar. 8, 2022

Related U.S. Application Data
(60) Provisional application No. 63/157,939, filed on Mar. 8, 2021.

(51) Int. Cl. C12M 1/00; C12M 1/36

(52) U.S. Cl. CPC C12M 23/52; C12M 41/48; C12M 23/34

(57) ABSTRACT

A mobile processing laboratory (MPL) is provided, configured for facilitating performing therewithin a cell therapy process. The MPL comprises a portable enclosure; one or more pieces of laboratory equipment for carrying out the cell therapy process and being housed within the enclosure; a plurality of sensors, each configured to measure information regarding the environment, cellular material of the process, and/or one of the pieces of laboratory equipment; and a computer system configured for management of the cell therapy process. The computer system is configured to facilitate collecting data from the sensors, and to optimize one or more activities associated with performance of the cell therapy process based on data collected from one or more other MPLs configured for performing therewithin substantially the same cell therapy process.

### CROSS REFERENCE TO RELATED APPLICATIONS

This application claims the benefit of U.S. Ser. No. 63/157,939, filed on Mar. 8, 2021, which is incorporated herein by reference in its entirety.

### TECHNOLOGICAL FIELD

The presently disclosed subject matter relates to laboratories for cell therapies, in particular to mobile laboratories.

### BACKGROUND

Cell therapy, which is designed to boost the immune response, represents a revolution in cancer treatment, especially for certain types of hematologic malignancies, as opposed to traditional cancer treatments such as chemotherapy and radiation. Despite the demonstrated benefits of currently approved cell therapies, they have substantial limitations, for example pertaining to the manufacturing process.

### SUMMARY

According to an aspect of the presently disclosed subject matter, there is provided a mobile processing laboratory (MPL) configured for facilitating performing therewithin a cell therapy process, the MPL comprising:
a portable enclosure;
one or more pieces of laboratory equipment for carrying out the cell therapy process and being housed within the enclosure;
a plurality of sensors, each configured to measure information regarding the environment, cellular material of the process, and/or one of the pieces of laboratory equipment;
a computer system configured for management of the cell therapy process, the computer system being configured to:
   facilitate collecting data from the sensors; and
   optimize one or more activities associated with performance of the cell therapy process based on data collected from one or more other MPLs configured for performing therewithin substantially the same cell therapy process.

A cell therapy process performed in another MPL may be considered to be substantially the same if the same steps are carried out on similarly classified cellular material with the goal of reaching the same endpoint. It will be appreciated that even though some of the steps may, in practice, differ between the two, the processes may still be considered to be substantially the same if the differences arose out of consideration of parameters of the cellular material and/or conditions during the process, i.e., that each process was carried out and/or altered in view of considerations which were common to both.

Cellular material may be considered to be similarly classified, e.g., if they each meet the requirements of the cell therapy processes.

The other MPLs may be configured for performing the cell therapy process on equipment substantially the same as the laboratory equipment.

The equipment of the two MPLs may be considered to be substantially the same if, e.g., they are not identical but carry out substantially similar processes in the same way, for example two centrifuges provided by different manufactures may be considered to be substantially the same as one another. Whether or not equipment is substantially the same may depend, e.g., on whether or not measurements and/or results of one may be used to inform management of the other, in particular with regards to optimization thereof.

The computer system may be configured to perform the optimization based on a machine learning algorithm.

The one or more activities may comprise management of rate of usage of the laboratory equipment.

The one or more activities may comprise management of supply chain logistics.

The one or more activities may comprise management of maintenance of the laboratory equipment.

The one or more activities may comprise management of replacement of the laboratory equipment.

The one or more activities may comprise management of coordination between the laboratory equipment.

At least some of the laboratory equipment may be selected from a group including a bio isolator, a cell manufacturing system, a bio safety cabinet, an incubator, a cell counting device, a microscope, an electroporator, an image analyzer, a refrigerator, a freezer, a liquid nitrogen tank, a sonication device, a UV chamber, a light table, a stereoscope, a shaking incubator, a peristaltic pump, a particle sampler, a sealer, a welder, a cell processing system, a centrifuge, a pipettor, a vortex mixer, and an X-ray irradiator.

At least some of the process sensors may be selected from a group including a dissolved oxygen sensor, a pH sensor, a process sensor, a lactate-glucose sensor, a temperature sensor, a metabolic sensor, a biomass sensor, an optical sensor, and a pressure sensor.

At least some of the environmental sensors may be selected from a group including a temperature sensor, a humidity sensor, a pressure, and a particle counter.

The interior of the enclosure may be divided into two or more rooms.

A first of the rooms may be connected by a doorway to the exterior of the MPL and maintained at a higher ambient pressure, with a last of the rooms being connected by a doorway to an adjacent room and maintained at a higher ambient pressure.

The interior of the enclosure may be further divided into additional rooms, each being connected by doorways to two of the rooms, the doorways defining a path between the first and last rooms, wherein each room is maintained at an ambient pressure which is higher than that of a room adjacent thereto being closer along the path to the first room.

The laboratory equipment may be housed within the last room.

The MPL may further comprise auxiliary laboratory equipment housed in at least one room other than the last room.

The MPL may be configured for connection to one or more externally supplied infrastructure services.

The infrastructure services may comprise one or more selected from a group including electric power, water supply, drainage, and gas supply.

Providing an MPL as above, in particular comprising a computer system configured to provide optimization, facilitates providing a network of substantially identical MPLs, each of which is a source of data about the cell therapy process performed thereby. As the MPLs of the network are identical and carry out substantially the same process, the optimization of all of the MPLs is greatly improved, as it is based on a large amount of data generated by the MPLs in the network and which is easily applicable to the processes carried out thereby. The MPLs of the network may communicate directly with one another, and/or they may communicate with a central computer system which carries out at least some of the optimization.

It will be appreciated that this leveraging of data from many MPLs is facilitated at least in part by the fact that providing the MPL as a standalone unit, i.e., within a portable enclosure, expedites mass-production and deployment of substantially identical MPLs. In contrast, laboratories which are each custom-built in a space allotted within a facility (for example in a hospital), and thus must conform to external constraints, are likely to be dissimilar from other custom-built laboratories, thereby impeding the use of data from each to optimize other laboratories.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a schematic top view of a mobile processing laboratory (MPL) according to the presently disclosed subject matter;

FIG. 1B is a side view of a bottom portion of the MPL illustrated in FIG. 1A;

FIGS. 1C through 1E are schematic top views of different examples of mobile processing laboratories according to the presently disclosed subject matter;

FIG. 2 is a schematic view of a bio-isolator of the MPL illustrated in FIG. 1A;

FIGS. 3A through 3C illustrated another example of a bio-isolator of the MPL illustrated in FIG. 1A;

FIG. 4A is a perspective view of a further example of a bio-isolator of the MPL illustrated in FIG. 1A;

FIG. 4B is a top view of the bio-isolator illustrated in FIG. 4A; and

FIG. 4C is a schematic illustration of flow through the bio-isolator illustrated in FIG. 4A.

### DETAILED DESCRIPTION

According to the presently disclosed subject matter, there is provided at least one mobile processing laboratory (hereinafter, “MPL”). Each MPL is a closed end-to-end manufacturing system which provides flexibility and scalability, enabling users to run one or more processes specified for a predetermined cell therapy. The MPL may be configured to produce multiple patient doses in parallel, for example several doses of the same therapy in a single MPL, therapies for different patients in a single MPL, different therapies and/or different portions/stages of a single therapy in separate MPLs, etc., thereby increasing manufacturing scalability. Moreover, use of one or more MPLs as per the presently disclosed subject matter may be associated with a reduced per-patient cost, for example when compared with producing the same and/or equivalent doses using known and/or conventional approaches.

The MPL 100 may be located in proximity to a medical center, thereby facilitating low-cost/high-quality solution for harmonized supply across a wide network.

The MPL 100 may be designed to fit in standard 40′ shipping container, e.g., having external dimensions of 2.4 m (8′−0″)×12.1 m (40′−0″)×2.8 m (9′−6″) (W×L×H), and internal dimensions of 2.2 m (7′−5″)×11.6 m (37′−11″)×2.3 m (7′−8″) (W×L×H). Accordingly, it may be transported by a standard semitrailer.

The MPL 100 may be provided to be connected to infrastructure provided by an external source (e.g., the medical center), for example including, but not limited to, electric power/backup, water, drainage, gasses, warehouse, and a quality control laboratory.

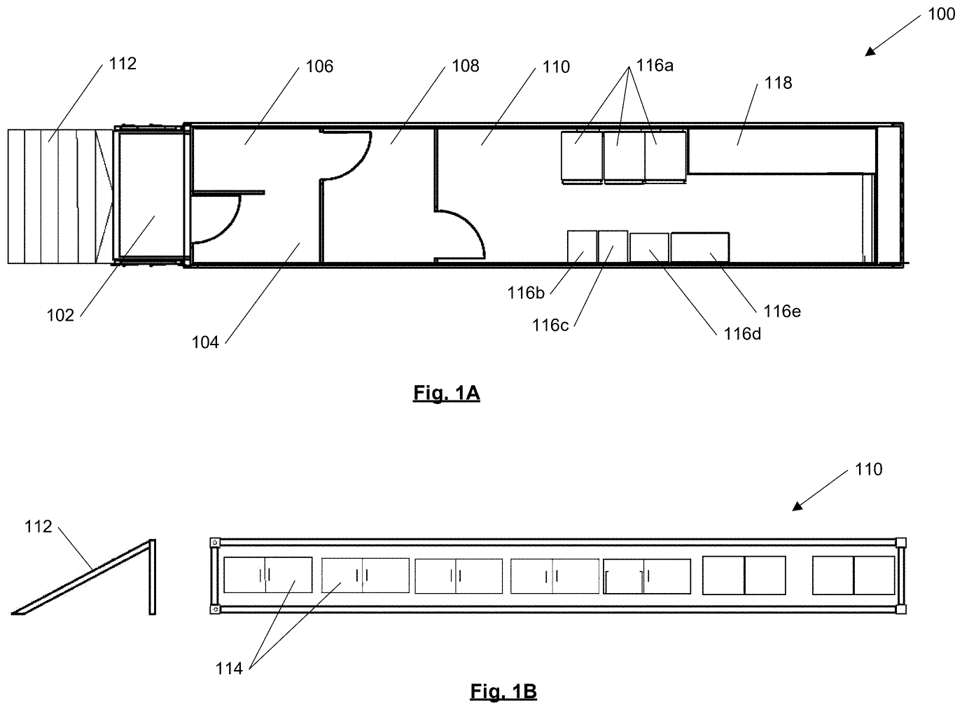

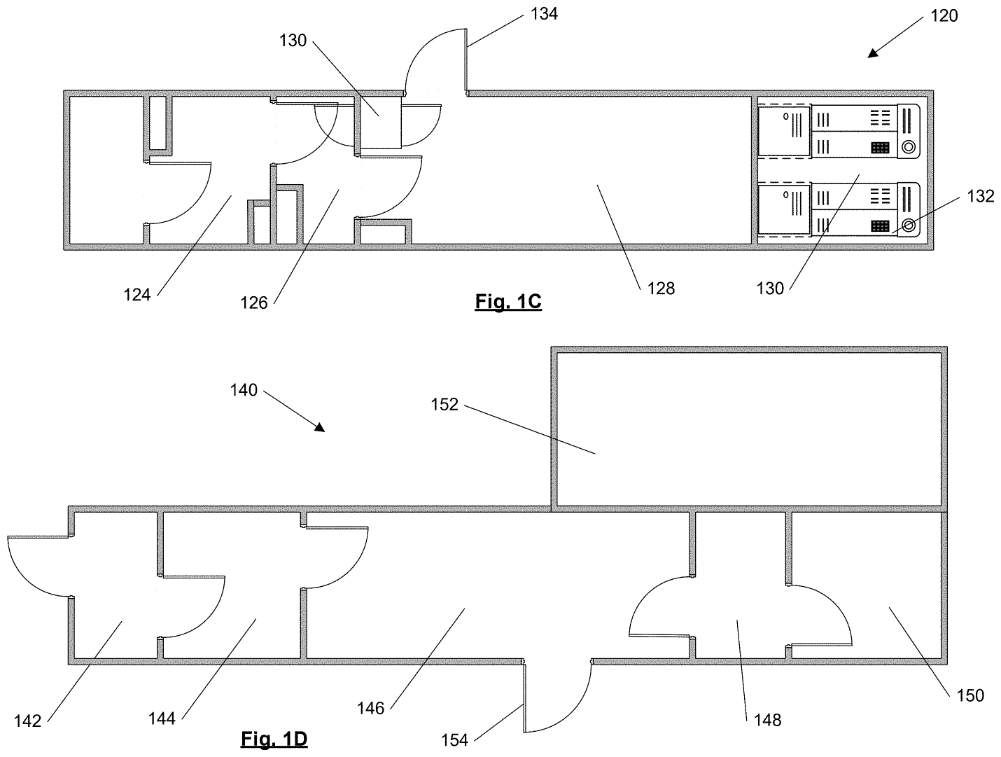

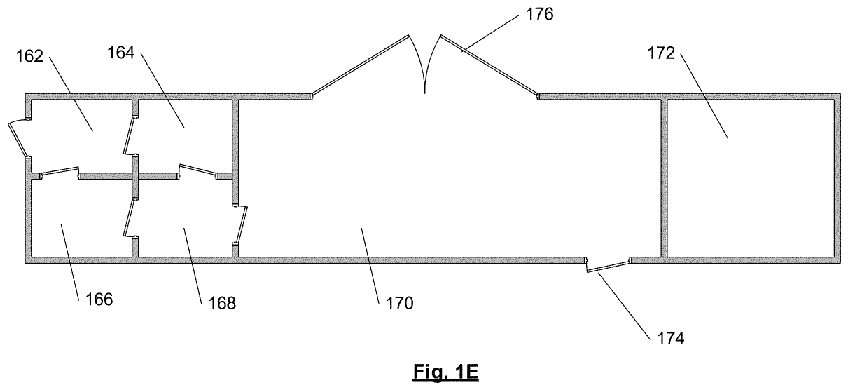

According to one example, for example as illustrated in FIG. 1A, an MPL is provided, generally indicated at 100. The MPL 100 comprises a dressing room 102, first and second storage rooms 104, 106, a preparation room 108, and a main processing room 110. The rooms may be connected to each other in order to control flow of personnel therethrough, i.e., the dressing room 102 comprises a doorway providing access to/from the exterior of the MPL 100; the dressing room 102 and the first storage room 104 are connected by a doorway providing access therebetween; the first storage room 104 and the second storage room 106 are connected by a doorway providing access therebetween; the second storage room 106 and the preparation room 108 are connected by a doorway providing access therebetween; and the preparation room 108 and the main processing room 110 are connected by a doorway providing access therebetween.

Each of the rooms may be maintained at a predetermined set of environmental conditions. For example:
the dressing room 102 may be maintained at temperature of about 20°±2° C., a relative humidity below about 65%, and a pressure which is slightly above the ambient pressure; the first storage room 104 may be maintained at temperature of about 20°±2° C., a relative humidity below about 65%, and a pressure which is slightly above that of the dressing room 102;
the second storage room 106 may be maintained at temperature of about 20°±2° C., a relative humidity below about 65%, and a pressure which is slightly above that of the first storage room 104;
the preparation room 108 may be maintained at temperature of about 20°±2° C., a relative humidity below about 65%, and a pressure which is slightly above that of the second storage room 106; and the main processing room 110 may be maintained at temperature of about 20°±2° C., a relative humidity below about 65%, and a pressure which is slightly above that of the preparation room 108.

It will be appreciated that the air pressure in each of the rooms increases towards the main processing room 110. Accordingly, when a doorway between rooms is open, airflow tends to be away from the main processing room 110.

Moreover, each of the rooms may be maintained so as to meet a predetermined standard, e.g., relating to the maximum concentration of particles in the air. For example, the dressing room 102 may meet the standards of Grade D classification according to the EU GMP for Manufacture of Sterile Medicinal Products, the first and second storage rooms 104, 106 may meet the standards of Grade C classification according to the EU GMP for Manufacture of Sterile Medicinal Products, and the preparation and main processing rooms 108, 110 may meet the standards of Grade B classification according to the EU GMP for Manufacture of Sterile Medicinal Products.

As illustrated in FIG. 1B, the rooms of the MPL 100 may be raised above the ground and accessible by stairs 112. Accordingly, a plurality of compartments, for example comprising one or more storage compartments 114, may be provided below the rooms.

Reverting to FIG. 1A, the main processing room 110 may be provided with one or more stations 116, each configured to facilitate performing one or more steps of a cell therapy process. According to some examples, the main processing room may comprise one or more incubator stations 116a, a scale station 116b (for example comprising a hook weight), an irradiation station 116c, and a centrifuge station 116d. The main processing room 110 may further comprise a workbench 118 comprising freestanding equipment, including, but not limited to, one or more selected form a particle measuring system, a counting device, a microscope, and/or a centrifuge.

It will be appreciated that herein the present description, base reference numerals may be used without trailing letters to collectively refer to all elements indicated thereby; accordingly, e.g., the term “stations 116” may be used to collectively refer to the incubator station 116a, scale station 116b, irradiation station 116c, and centrifuge station 116d, and/or to a subset thereof.

According to some examples, the main processing room 110 may comprise other equipment, in place of and/or in addition to some or all of that described above with reference to and as illustrated in FIG. 1A.

According to some examples, an MPL 120 may be provided as illustrated in FIG. 1C. According to these examples, the MPL 120 may comprise a dressing room 122 (which may comprise, e.g., one or more stepovers with or without compartments, and/or any other suitable accouterments), a storage room 124 connected thereto, a preparation room 126 connected thereto, and a main processing room 128 connected thereto. A pass box 130 may be provided between the preparation room 126 and the main processing room 128.

Similar to as described above, each of the rooms may be maintained at an air pressure which is above that which precedes it, such that when a doorway between rooms is open, airflow tends to be away from the main processing room 128.

The MPL 120 may further comprise a technical room 132, which is not directly accessible via any of the other rooms of the MPL, and which may house air-handling units 134 and/or other suitable equipment for providing services to the MPL, including, but not limited to, control/monitoring systems, an autoclave, and/or a decontamination/sterilization system.

The main processing room 128 may be provided with an emergency exit 136, e.g., which is configured only to be opened from the inside. According to some examples, the MPL 120 may be configured to take predetermined actions if the emergency door is opened, for example to prevent contamination of the cell therapies being processed.

According to some designs, an MPL 140 may be provided as illustrated in FIG. 1D. According to these examples, the MPL 140 may comprise a first dressing room 142, a preparation room 144 connected thereto, a main processing room 146 connected thereto, a second dressing room 148 connected thereto, and a biological safety cabinet 150 connected thereto. According to some examples, the main processing room may meet the standards of Grade C classification according to the EU GMP for Manufacture of Sterile Medicinal Products, and the second dressing room 148 and biological safety cabinet 150 are classified as Grade B.

The MPL 140 may comprise a technical room 152, and/or the main processing room 146 may be provided with an emergency exit 154, each similar to as described above with reference to FIG. 1C.

According to some designs, an MPL 160 may be provided as illustrated in FIG. 1E. According to these examples, the MPL 160 may comprise a locker room 162 connected to a material airlock 164 and a personnel airlock 166. The personnel airlock 166 is further connected to a sterile gowning room 168, which is connected to the main processing room 170.

The MPL 160 may comprise a technical room 172, and/or the main processing room 170 may be provided with an emergency exit 174, each similar to as described above with reference to FIG. 1C. The MPL 160 may further comprise large access doors 176 for providing access to the equipment from outside the MPL, for example for maintenance. Providing direct access to equipment may facilitate maintenance without undue risk of contaminating the other rooms of the MPL.

It will be appreciated that the names of the rooms provided herein are representative of one use thereof, and in practice a room may be configured for performing therein a function which differs from what is suggested by its name.

It will be further recognized that reference numeral used in FIGS. 1A through 1E are for convenience of describing the different examples. Accordingly, only one set of reference numerals will be used in the presently disclosed subject matter, but should be construed as being relevant to each of the examples, mutatis mutandis (e.g., a reference herein to an “MPL 100” or a “main processing room 110,” for example relating to its structure, use, and/or equipment provided therein, is applicable as well, in part or in whole as appropriate, to any of the examples described above with reference to and as illustrated in FIGS. 1C through 1E, unless otherwise clear from context).

According to some examples, one or more cell manufacturing systems are provided in the main processing room 110. Examples of such system include, but are not limited to:
Adva X3, sold by ADVA Biotechnology of Bar Lev Industrial Park, Israel;
Aglaris Facer sold by by Aglaris, Ltd. Of Stevenage, England;
Cocoon® platform sold by Lonza Group of Basel, Switzerland;
CliniMACS Prodigy® sold by Miltenyi Biotec of Bergisch Gladbach, Germany;
a cell manufacturing platform provided by Oribiotech, Ltd., of London, England and Woodcliff Lake, N.J.;
Quantum® Cell Expansion System sold by Terumo Corporation of Tokyo, Japan; and
a cell manufacturing/expansion system sold by VivaBioCell S.p.A. of Udine, Italy;

The main processing room 110 may further comprise any one or more of the following:
a biosafety cabinet (for example as sold by Thermo Fisher Scientific of Waltham Mass. or by Esco Group of Singapore;
an incubator;
a cell counting device (for example NucleoCounter® NC-200™ sold by ChemoMetec of
Allerod, Denmark);
a microscope;
an electroporator;
an image analyzer;
a refrigerator;
a freezer (for example as sold by Thermo Fisher Scientific);
a liquid nitrogen tank;
a sonication device;
a UV chamber;
a computer/tablet;
a light table;
a stereoscope;
a shaking incubator;
a human-machine interface (HMI) control panel;
a peristaltic pump (for example as sold by Watson-Marlow Fluid Technology Group of Falmouth, England);
a particle sampler (for example as sold by Particle Measuring Systems of Boulder, Colo.);
a sealer (for example as sold by Fresenius Kabi of Bad Homburg, Germany);
a welder (for example as sold by Fresenius Kabi);
a cell processing system (for example as sold under the trademark Lovo® by Fresenius Kabi);
a centrifuge (for example as sold by Thermo Fisher Scientific);
an incubator (for example as sold by Thermo Fisher Scientific);
a pipettor (for example as sold by Thermo Fisher Scientific);
a pH meter (for example as sold by Mettler Toledo of Columbus, Ohio);
an analytical balance (for example as sold by Mettler Toledo);
a vortex mixer (for example as sold by Heidolph Instruments of Schwaback, Germany);
a cell culture microscope (for example as sold by Nikon Corporation of Tokyo, Japan);
an X-ray irradiator (for example as sold by Faxitron Bioptics, LLC of Tucson, Ariz.);
a spin down centrifuge (for example as sold by Alex Red, Ltd. of Mevasserret-Zion, Israel); and/or
a hook scale (for example as sold by Barmaper Dan, of Acre, Israel)

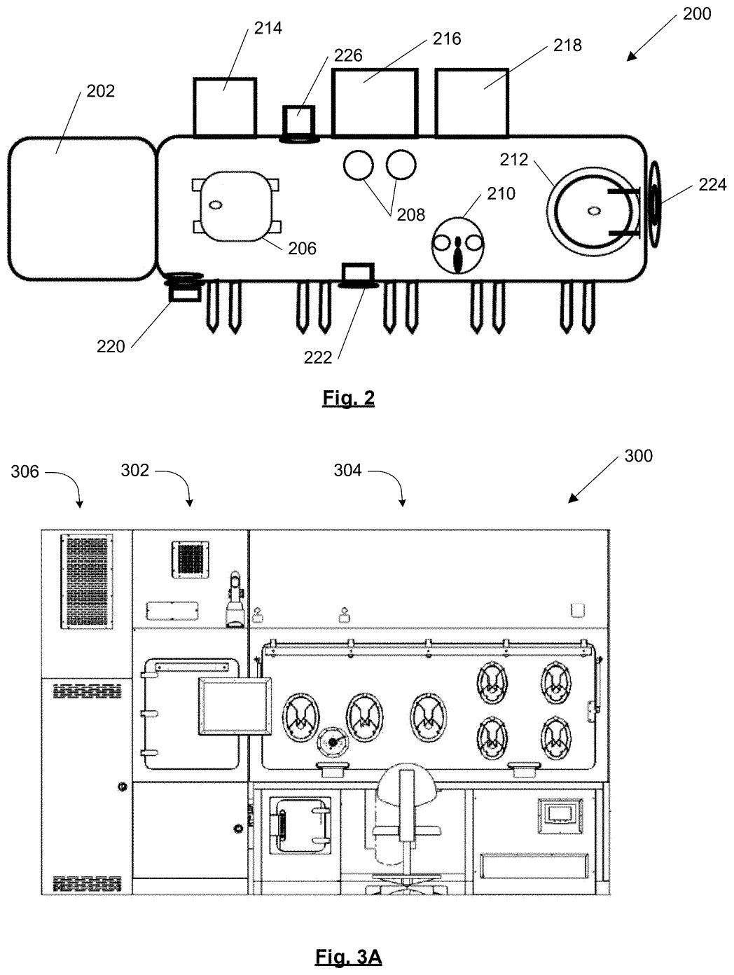

For example, as illustrated in FIG. 2, the main processing room 110 may comprise a bio-isolator 200. The bio-isolator 200 may be an advanced therapy medicinal product (ATSM) standalone unit characterized by one or more of the following:
it may meet the International Organization for Standards ISO-5 classification for cleanrooms;
it may provide continuity throughout which meets the standards of Grade A classification according to the EU GMP for Manufacture of Sterile Medicinal Products;
it may comprise one or more HEPA H14 filters, for example providing laminar flow therethrough;
positive pressure cascade regime;
HEPA H14 filters for exhaust;
vaporized H₂O₂ bio-decontamination
a compliant GxP computerized system, for example per the International Society for Pharmaceutical Engineering GAMP® 5 Guide; and
sash window panels, for example having a 10° slope.

The bio-isolator 200 may comprise, inter alia, a pre-chamber 202 comprising a laminar flow hood (LFH) and being in selective communication (i.e., connected thereto by an opening which may be selectively opened and closed, thereby allowing, e.g., a user to temporarily allow access therebetween) with a process chamber 204. The process chamber 204 may comprise a hatch 206, e.g., for removal of waste, one or more one-way hatches 208, a glove port 210, and a centrifuge 212, and a storage compartment 214. It may further comprise an incubator 216 and/or a refrigerator 218. A sterile liquid access port 220, mousehole port 222, a rapid transfer port 224, and/or a pump 226 may further be provided.

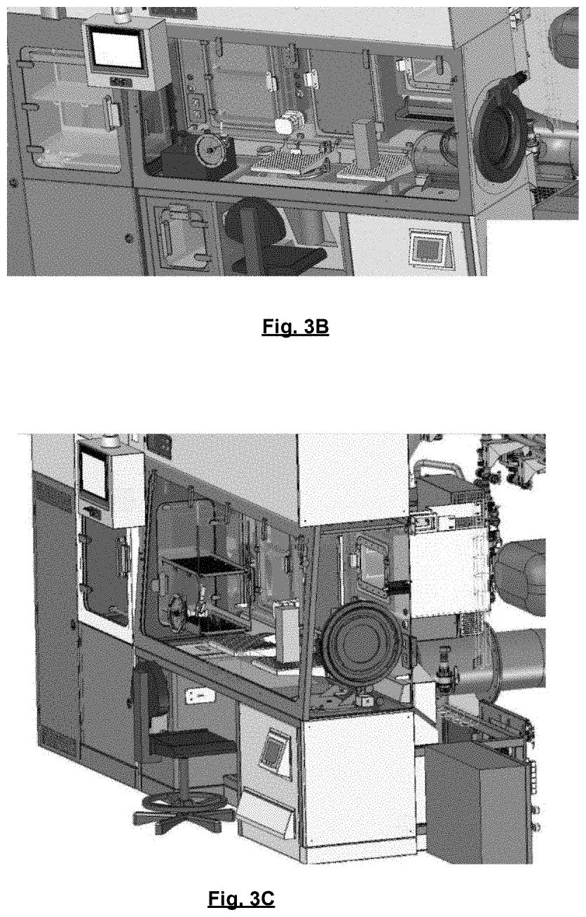

According to another example, such as is illustrated in FIGS. 3A through 3C, a bio-isolator 300 may be provided, comprising, inter alia, a pre-chamber 302 in selective communication with a process chamber 304, and a vapor-phase H₂O₂ cabinet 306.

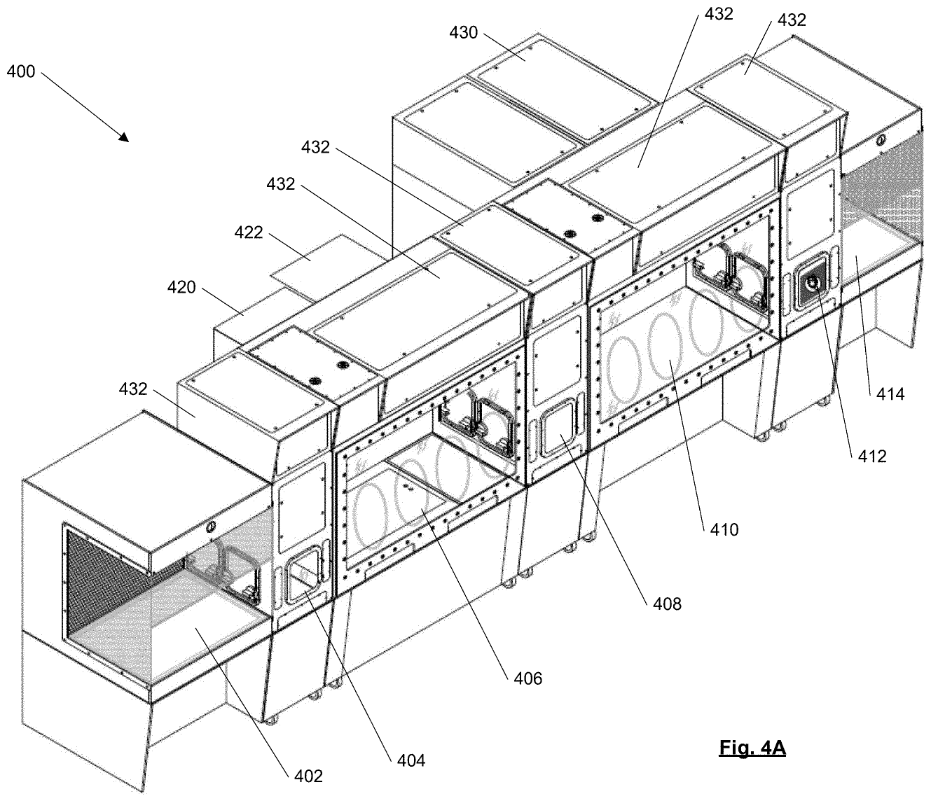

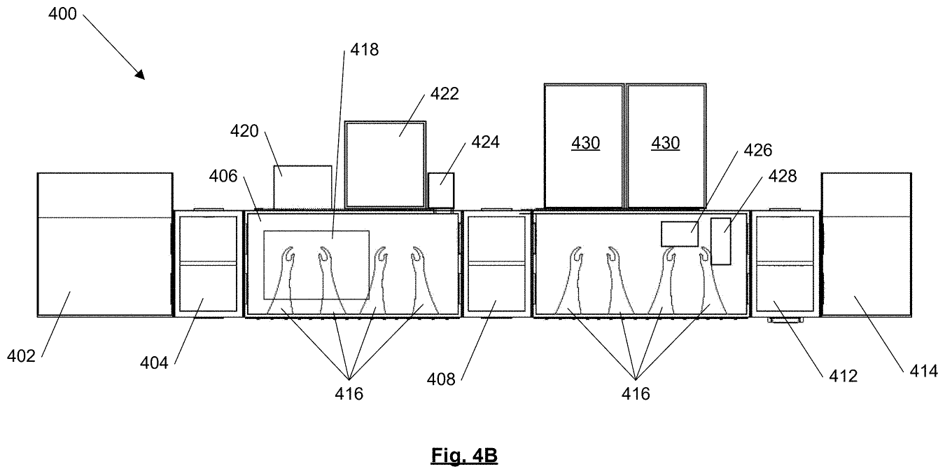

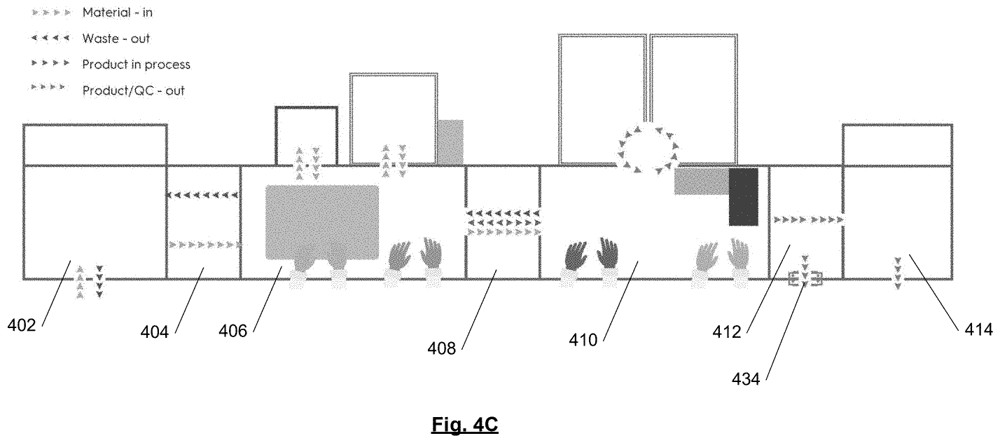

According to another example, such as is illustrated in FIGS. 4A and 4B, a bio-isolator 400 may be provided. The bio-isolator 400 is configured to prevent intermixing of room air with air witching processing chambers thereof, and release of air therefrom into the room. According to some examples, all therewithin is provided from an external source of sterile air, and all air removed therefrom is sterilized and/or filtered before being released external to the MPL 100.

The bio-isolator 400 provides a suitably low humidity environment and allows for necessary gas changing. It is a closed system, meeting the International Organization for Standards ISO-5 classification for cleanrooms, even when the exterior thereof meets a lower standard, e.g., ISO-8 classification for cleanrooms. It is modular, facilitating scaling up and/or down as necessary. It may be provided with and/or be configured to interface with a computer system, thereby facilitating control, maintaining of batch records, etc.

The computer system may be compliant with one or more regulations, for example those required by the US Food and Drug Administration for electronic records (e.g., as per the Code of Federal Regulations of the United States, Title 21, § 11) and/or as per the International Society for Pharmaceutical Engineering GAMP® 5 Guide. The computer system may be configured to provide system security (e.g., password protection, administrator tools, safety protocols, logout and lockout options, etc.), audit trails (e.g., time-stamped event logs, marking changes as “old,” “new,” etc.), securely storing all data files, graphs, etc., on a storage device.

The computer system may be configured to optimize activities performed within the MPL 100. For example, it may collect data, e.g., from one or more sensors within the MPL 100, from users regarding processing being performed therein, patient data, etc., and perform one or more optimization based at least partially thereon. The sensors may include, but are not limited to:
process sensors, e.g., dissolved oxygen, pH, lactate-glucose, temperature, metabolic sensor, biomass sensor, optical sensor (such as a microscope), gas and liquid pressure, etc.; environmental sensors, e.g., temperature, humidity, pressure, particle counter (viable and/or non-viable), etc.; and/or
machine sensors, e.g., monitoring one or more bioreactors, centrifuges, incubators, mixing chambers, QC, peristaltic pumps, barcode readers, batch records, etc.

The optimizations may include, but are not limited to:
rate of usage, i.e., when to begin the next cell therapy, thereby maximizing the number of patients being treated in a given amount of time;
supply optimization, i.e., statuses of materials, machines, consumables, etc., thereby optimizing supply chain logistics, maintenance, repair/replacement, etc.; and/or
MPL optimization, i.e., facilitating synchronization between processes, equipment, automation, QC, and MPLs 100, for example to facilitate parallel and/or coordinated processing.

The optimizations may be performed based on one or more predetermined algorithms, data collected from previous cell therapies (e.g., big data, for example including data relating to processes, sensors, patients, etc.), and/or a machine learning algorithm. This may facilitate:
increasing the quality of therapies and/or process robustness;
facilitating parallel and/or shorter processes thereby optimizing processing;
facilitating data collaboration among different members of, e.g., health networks;
facilitating provisioning materials and/or supplies to one or more MPLs, e.g., thereby implementing a “just-in-time” supply chain, for example based on one or more of anticipated need, inventory of other MPLs, etc.;
reducing preparation time for a subsequent therapy or process step, thereby increasing MPL efficiency;
anticipating when a process step will complete and a subsequent one may begin;
prediction of when processing of a therapy will be completed;
adjusting the cleanroom grades according to need; and
providing recommendations for allocation of resources, for example where to install MPLs, which systems existing or future MPLs should contain, etc.

According to some examples, the computer system may be configured to perform an optimization algorithm to determine when a patient should begin preparation stages of a cell therapy process to be performed within the MPL 100, in particular by automated equipment therewithin. More particularly, the algorithm may be used, e.g., when the equipment of the MPL 100 supports a process which is fully automated, with the exception of an initial step. The automated portion of the process is performed using one or more pieces of equipment which are considered by the computer system, at least for the purpose of executing the algorithm, as one or more “automatic units.” The non-automated initial step is performed using one or more pieces of equipment which are collectively considered by the computer system, at least for the purpose of executing the algorithm, as a “manual unit.”

According to some examples, the computer system is configured to perform the algorithm to optimize a situation in which there is a single manual unit. The MPL 100 may support performing a single automated process, or multiple automated processes simultaneously, by the automatic units. As noted, each of the automated processes begins with a step or steps performed in the manual unit; similarly, some or all of the automated processes may require that an additional step or steps, for example a final step, be performed using the manual unit.

In a first step of the algorithm, the computer system determines if any automatic units (i.e., one or more pieces of equipment which operate together and/or in a predefined sequence to perform an automated portion of a cell therapy process) are available for use. This may be performed by gathering information about the automatic units in the MPL 100, for example if the total number of automatic units in the MPL 100 exceeds the number of automatic units in use (i.e., engaged in a cell therapy process).

It will be appreciated that while the optimization algorithm is described with reference to a single MPL 100, this is by way of example only, and in practice it may be performed across a plurality of MPLs, for example considering each MPL to be a single automatic unit and/or by considering all of the automatic units in the plurality of MPLs together, mutatis mutandis.

In the case in which the first step of the algorithm returns a positive value (i.e., at least one automatic unit is available for use), the algorithm continues along a first “yes-branch,” in which the computer system determines whether or not the manual unit is currently available. The algorithm proceeds as follows:
If the manual unit is currently available, the computer system determines the “new patient manual time,” i.e., the amount of time required to complete the manual process for a new patient, which includes the time required for any preparatory steps to obtain a sample, plus the time needed to process the sample in the manual unit, as well as the “minimum AU delivery time,” i.e., the expected minimum amount of time until any one of the automatic units currently processing a sample will need to return a sample to the manual unit for further processing, for example as described above.
   i. If the new patient manual time exceeds the minimum AU delivery time (i.e., if at least one currently running automated processes will require that its sample be processed in the manual unit before a sample from a new patient can be obtained and the initial manual step completed), no new patient should begin preparatory steps for obtaining a sample. The computer system may indicate as such and/or that the manual unit is awaiting delivery of a sample from an automatic unit.
   ii. If the new patient manual time does not exceed the minimum AU delivery time (i.e., a sample from a new patient can be obtained and the initial manual step completed before any of the currently running automated processes will require that its sample be processed in the manual unit before), then preparatory steps for obtaining a sample from a new patient may begin. The computer system may indicate as such.
If the manual unit is currently unavailable (i.e., it is currently processing a sample), no new patient should begin preparatory steps for obtaining a sample. The computer system may indicate that the manual unit is occupied. According to some examples, the computer system further determines if the sample being processed in the manual unit is a new sample (i.e., one which is undergoing the initial step), or if it is being finalized (i.e., if it has already undergone the automated process), and may additionally indicate such.

In the case in which the first step of the algorithm returns a negative value (i.e., none of the automatic units is available for use), the algorithm continues along a first “no-branch,” in which the computer system determines whether or not the manual unit is currently available.
If the manual unit is available, no new patient should begin preparatory steps for obtaining a sample, as no automatic units are available. The computer system may indicate that the manual unit is available but that no automatic units are.
If the manual unit is not available, no new patient should begin preparatory steps for obtaining a sample. According to some examples, the computer system further determines if the sample being processed in the manual unit is a new sample (i.e., one which is undergoing the initial step), or if it is being finalized (i.e., if it has already undergone the automated process), and may indicate such.

It will be appreciated that irrespective of the state of the manual unit, there are no circumstances under the first “no-branch” in which a new patient should begin preparatory steps for obtaining a sample. However, the status id the manual unit is still checked. While the result of this check doesn't impact the current iteration of the algorithm, the information may be useful for other purposes, e.g., data which may be used to develop models for future optimizations, etc.

It will be further appreciated that the above represents non-limiting examples of an algorithm which may be used to optimize the system. The computer system may be configured to perform modifications thereof and/or other algorithms to optimize the system, including, but not limited to, algorithms which are configured to optimize other parameters.

The bio-isolator 400 may comprise a plurality of modules. According to some examples, the modules include, but are not limited to, a first laminar flow clean hood 402 in selective communication with the exterior of the bio-isolator 400, a first buffer chamber 404 in selective communication with the first laminar flow clean hood, a first processing chamber 406 in selective communication with the first buffer chamber, a second buffer chamber 408 in selective communication with the first processing chamber, a second processing chamber 410 in selective communication with the second buffer chamber, a third buffer chamber 412 in selective communication with the second processing chamber, and a second laminar flow clean hood 414 in selective communication with the third buffer chamber and with the exterior of the bio-isolator.

In order to facilitate an MPL 100 of a suitably small size, e.g., to fit within a 40′ shipping container for example as described above, the size of each of the modules of the bio-isolator 400 may be minimized. For example:
the first laminar flow clean hood 402 may have a width of approximately 3′;
the first buffer chamber 404 may have a width of approximately 18″;
the first processing chamber 406 may have a width of approximately 57″;
the second buffer chamber 408 may have a width of approximately 18″;
the second processing chamber 410 may have a width of approximately 57″;
the third buffer chamber 412 may have a width of approximately 18″; and
the second laminar flow clean hood 414 may have a width of approximately 2′.

The first processing chamber 406 may comprise one or more glove ports 416, and a centrifuge 418, e.g., disposed in a compartment below a working area thereof. It may further comprise a storage unit 420, a refrigerator 422, and a particle counter 424.

The second processing chamber 410 may comprise one or more glove ports 416, a microscope 426 and a peristaltic pump 428. It may further comprise one or more incubator bank modules 430.

It will be appreciated that the elements within the processing chambers 406, 410 is by way of example only, and in practice any suitable combination of elements, including, but not limited to, some or all of those described above, may be provided.

For example, one or more automated and closed cell expansion systems may be provided. Such systems may be configured to automatically perform some or all processing steps from tissue/fluid isolation through end-product production. They may be configured for one or more of CAR-T, TIL, and NK immune cell production.

A plurality of compartments 432 may be disposed above some or all of the modules. The compartments 432 may comprise one or more storage compartments, controller enclosure, air conditioning units, etc.

As illustrated in FIG. 4C, in use the bio-isolator 400 may be configured to accommodate flow (e.g., be moved by a user) of different types of materials in different direction between the modules thereof. For example:
raw material may flow into the first laminar flow clean hood 402 from the exterior of the bio-isolator 400;
waste may flow out of the first laminar flow clean hood 402 to the exterior of the bio-isolator 400;
raw material may flow from the first laminar flow clean hood 402 into the first processing chamber 406 via the first buffer chamber 404;
waste may flow from the first processing chamber 406 to the first laminar flow clean hood 402 via the first buffer chamber 404;
raw material may flow between the first processing chamber 406 and the storage unit 420 associated therewith;
raw material may flow between the first processing chamber 406 and the refrigerator 422 associated therewith;
raw material and product-in-process may flow from the first processing chamber 406 into the second processing chamber 410 via the second buffer chamber 408;
waste and product-in-process may flow from the second processing chamber 410 into the first processing chamber 406 via the second buffer chamber 408;
final product (including samples for quality control) may flow between the second processing chamber 406 and one or both of the incubator bank modules 430 associated therewith;
final product (including samples for quality control) may flow from the second processing chamber 406 to the exterior of the bio-isolator 400 via the third buffer chamber 412 and the second laminar flow clean hood 414; and/or
final product (including samples for quality control) may flow from the second processing chamber 406 to the exterior of the bio-isolator 400 via the third buffer chamber 412 and a connector 434 which facilitates aseptic liquid transfer therethrough, for example a SART System™ port marketed by Sartorius AG.

The MPL 100 may be configured to provide, wholly or in part, any suitable process and/or steps of a cell therapy regime. According to some examples, it may be configured to perform some or all steps of a CART (chimeric antigen receptor T-cells) therapy, including, but not limited to, PBMC (peripheral blood mononuclear cell) extraction, cell sorting, activation, transduction, expansion in bioreactor, harvesting, and/or washing.

According to further examples, it may be configured to perform some or all steps of a MOTC (metabolic optimized T-cells) therapy, including, but not limited to, steps which are performed to obtain a first-stage, sometimes referred to as pre-REP (rapid expansion protocol) product, such as washing and seeding a tumor sample collected from a patient, replacement of media as necessary (for example in view of the glucose-lactate ratio thereof), harvest and cryopreservation, etc., as well as steps which are performed to generate a final product following a rapid expansion protocol, including, but not limited to, thawing, preparation of feeder cells, seeding, addition of media (for example per a glucose-lactate ratio thereof), harvesting, final formulation, quality control (e.g., evaluating cell count and/or viability), etc.

A typical process for drug formulation in the MPL 100, taking into account, inter alia, the patient's condition, doctors' instructions, and/or physical parameters/constraints, may include, but is not limited to:
performing steps in separate closed systems;
deciding, for example based on results of a machine learning algorithm such as described above, whether to advance to a subsequent step in the process;
facilitate transferring of cells to a system for a subsequent process step;
alerting an operator if a predetermined alarm condition, e.g., low yield, supplies needed, deviation from a set parameter, etc., has occurred;
performing automatic recovery or recommendation to an operator, e.g., to revert the process to a previous stage, to change the media, to add materials such as nutrition, activation, and/or transduction materials thereto, to change one or more process parameters (including, but not limited to, dissolved oxygen level, pH, glucose level, liquid level, RPM, and/or perfusion rate; and/or
deciding the number of gas-permeable culture device wells, for example as sold under the trade name G-REX® by Wilson Wolf Manufacturing of New Brighton, Minn., to use, for example in a MOTC process.

It will be appreciated than an MPL 100 as described herein with reference to and as illustrated in the accompanying drawings may facilitate providing a decentralized cell and gene therapy (CGT) supply chain. For example, as an end-to-end solution, it may provide:
multipurpose mobile autonomous CGT which conforms to good manufacturing processes (GMP);
proximity of a CGT supply chain to a medical center;
processing of multiple therapies simultaneously;
fast (e.g., 3-6 months) setup time;
small footprint;
viral and non-viral processing; and/or
large number of treatments at a relatively low cost.

Those skilled in the art to which this invention pertains will readily appreciate that numerous changes, variations, and modifications can be made without departing from the scope of the presently disclosed subject matter, mutatis mutandis.

1. A mobile processing laboratory configured for facilitating performing therewithin a cell therapy process, the mobile processing laboratory comprising:
a portable enclosure;
one or more pieces of laboratory equipment for carrying out the cell therapy process and being housed within said enclosure;
a plurality of sensors, each configured to measure information regarding the environment, cellular material of the process, and/or one of said pieces of laboratory equipment;
a computer system configured for management of said cell therapy process, said computer system being configured to:
facilitate collecting data from said sensors; and
optimize one or more activities associated with performance of said cell therapy process based on data collected from one or more other mobile processing laboratories configured for performing therewithin substantially the same cell therapy process.

2. The mobile processing laboratory according to claim 1, wherein said other mobile processing laboratories are configured for performing the cell therapy process on equipment substantially the same as said laboratory equipment.

3. The mobile processing laboratory according to claim 1, wherein the computer system is configured to perform the optimization based on a machine learning algorithm.

4. The mobile processing laboratory according to claim 1, wherein said one or more activities comprises management of rate of usage of said laboratory equipment.

5. The mobile processing laboratory according to claim 1, wherein said one or more activities comprises management of supply chain logistics.

6. The mobile processing laboratory according to claim 1, wherein said one or more activities comprises management of maintenance of said laboratory equipment.

7. The mobile processing laboratory according to claim 1, wherein said one or more activities comprises management of replacement of said laboratory equipment.

8. The mobile processing laboratory according to claim 1, wherein said one or more activities comprises management of coordination between said laboratory equipment.

9. The mobile processing laboratory according to claim 1, wherein said at least some of said laboratory equipment is selected from a group including a bio-isolator, a cell manufacturing system, a biosafety cabinet, an incubator, a cell counting device, a microscope, an electroporator, an image analyzer, a refrigerator, a freezer, a liquid nitrogen tank, a sonication device, a UV chamber, a light table, a stereoscope, a shaking incubator, a peristaltic pump, a particle sampler, a sealer, a welder, a cell processing system, a centrifuge, a pipettor, a vortex mixer, and an X-ray irradiator.

10. The mobile processing laboratory according to claim 1, wherein at least some of the process sensors are selected from a group including a dissolved oxygen sensor, a pH sensor, a process sensor, a lactate-glucose sensor, a temperature sensor, a metabolic sensor, a biomass sensor, an optical sensor, and a pressure sensor.

11. The mobile processing laboratory according to claim 1, wherein at least some of the environmental sensors are selected from a group including a temperature sensor, a humidity sensor, a pressure, and a particle counter.

12. The mobile processing laboratory according to claim 1, the interior of said enclosure being divided into two or more rooms.

13. The mobile processing laboratory according to claim 12, a first of said rooms being connected by a doorway to the exterior of the mobile processing laboratory and maintained at a higher ambient pressure, and a last of said rooms being connected by a doorway to an adjacent room and maintained at a higher ambient pressure.

14. The mobile processing laboratory according to claim 13, the interior of the enclosure being further divided into additional rooms, each being connected by doorways to two of said rooms, said doorways defining a path between said first and last rooms, wherein each room is maintained at an ambient pressure which is higher than that of a room adjacent thereto being closer along said path to said first room.

15. The mobile processing laboratory according to claim 13, wherein said laboratory equipment is housed within said last room.

16. The mobile processing laboratory according to claim 15, further comprising auxiliary laboratory equipment housed in at least one room other than said last room.

17. The mobile processing laboratory according to claim 1, being configured for connection to one or more externally supplied infrastructure services.

18. The mobile processing laboratory according to claim 17, wherein said infrastructure services comprise one or more selected from a group including electric power, water supply, drainage, and gas supply.
